(19) United States
(12) Patent Application Publication
(10) Pub. No.: US 2011/0070296 A1
(43) Pub. Date: Mar. 24, 2011

(54) TOPICAL COMPOSITIONS AND METHODS FOR UTILIZING PEPTIDES CONTAINING LIPID-MODIFIED CYSTEINE-CONTAINING PEPTIDES

(75) Inventors: Seung-Yub Lee, Palisades Park, NJ (US); Eduardo Perez, Somerset, NJ (US); Gopal Sarngadharan, Robbinsville, NJ (US); Jeffry B. Stock, Princeton, NJ (US); Maxwell Stock, Rocky Hill, NJ (US); Michael Voronkov, Pennington, NJ (US); Peter Wolanin, Princeton, NJ (US)

(73) Assignee: SIGNUM BIOSCIENCES, INC., Monmouth Junction, NJ (US)

(21) Appl. No.: 12/919,449

(22) PCT Filed: Feb. 25, 2009
(86) PCT No.: PCT/US09/35162

§ 371 Date: Dec. 6, 2010

Related U.S. Application Data
(60) Provisional application No. 61/067,161, filed on Feb. 25, 2008.

(51) Int. Cl. A61K 9/127; A61K 38/02; A61K 38/16; A61K 8/64; A61K 38/22; A61Q 11/00; A61P 17/00; A61P 17/10; A61P 39/00; A61P 17/12; A61Q 17/04; A61P 31/00; A61P 31/10; A61P 31/12; A61P 33/02; A61P 23/00; A61P 29/00; A61P 17/04; A61P 39/06; A61P 3/02; A61Q 19/00; A61Q 19/10

(52) U.S. Cl. 424/450; 514/1.1; 514/21.4; 424/49; 424/59; 514/9.7

(57) ABSTRACT

Described are compositions for topical application to keratinous tissues or substrates such as skin, or mucosal surfaces. The composition may be used to treat a skin condition or disease. The composition comprises at least one polypeptide and/or lipidated peptide and a carrier.

### RELATED APPLICATIONS

This application claims priority to U.S. provisional patent application Ser. No. 61/067,161, filed Feb. 25, 2008, the entire disclosure of which is incorporated herein by reference.

### BACKGROUND

Notwithstanding a desire for healthy skin, consistent exposure to elements exterior to the body causes damage to skin. Such damage may be caused by wounding, photodamage, atrophy, dry skin, etc. Presently, many lotions and creams that are in the market are purported to help alleviate skin damage and/or associated discomfort. Intensive efforts have continued towards the discovery of novel agents to treat and alleviate skin damage.

### SUMMARY

Accordingly, a first aspect of the present invention is directed to an isolated peptide or mixture of peptides having a lipidated cysteine residue, wherein the lipid is a polyisoprenoid, and a composition comprising the peptide or mixture thereof and a carrier suitable for topical administration. The term “topical administration” is meant to include direct or indirect application to a keratinous substrate such as skin, hair or scalp, or to mucosa (e.g., oral administration).

A second aspect of the present invention is directed to a method of promoting healthy skin or alleviating skin damage due to exposure to the elements, comprising applying to skin, directly or indirectly a composition comprising a peptide or mixture of peptides having a lipidated cysteine residue, wherein the lipid is a polyisoprenoid, and a carrier suitable for topical administration.

A third aspect of the present invention is directed to a method of making a topical composition for promoting healthy skin or alleviating skin damage due to exposure to the elements, comprising formulating a composition comprising a peptide or mixture of peptides having a lipidated cysteine residue, wherein the lipid is a polyisoprenoid, and a carrier suitable for topical administration.

In some embodiments of these aspects of the present invention, the sequence of a naturally occurring peptide or mixture of peptides contains a cysteine, whereas in other embodiments, a lipidated cysteine is introduced into the peptide.

In some embodiments, the peptide is about 2 to 200 amino acids in length while in other embodiments the mixture of peptides have an average size of about 2 to about 200 amino acids.

In some embodiments, a lipidated cysteine residue is present at the C-terminus. In some embodiments, a lipidated cysteine residue is at least one of the 5 C-terminal amino acid residues. In some embodiments, the peptide contains more than one lipidated cysteine residue, wherein two of the more than one cysteine residues are contiguous or non-contiguous, and wherein the C-terminus of the peptide may or may not be a lipidated cysteine residue. In some embodiments, the peptide contains a non-C-terminal lipidated cysteine residue. In some embodiments, the mixture of peptides may contain cysteine residues at varying amino acid positions in a lipidated peptide.

In some embodiments, the lipid group present on the cysteine is a geranyl, farnesyl, phytyl, or geranylgeranyl group, such that the lipidated residue is referred to as geranyl cysteine (GC) farnesyl cysteine (FC) phytyl cysteine (PC), and geranylgeranyl cysteine (GGC), respectively. In some embodiments, the peptide contains more than one lipidated cysteine residue wherein the lipid group is the same or different on each cysteine.

In some embodiments, the peptide is present in an amount of about 0.01 to about 10% w/v of the composition.

In some embodiments, the carrier is aqueous-based and the composition is in the form of an emulsion such as a gel or lotion. In some embodiments, the carrier is non-aqueous based (wherein aside from water that may be present in a commercially available element of the composition, the composition otherwise contains no added water), and the composition is in the form of an ointment or paste. In some embodiments, the composition is suitable for oral use.

In some embodiments, a pure lipidated peptide is blended with an additional carrier such as hydrolyzed silk protein to result in a uniformly lipid modified peptide. In some embodiments, a uniformly lipid modified peptide is formulated into a hair conditioner. In some embodiments, the hair conditioner contains a base. In some embodiments, the hair conditioner contains water. In some embodiments, the hair conditioner contains cetearyl alcohol.

### DETAILED DESCRIPTION OF CERTAIN EMBODIMENTS

Topical compositions may contain a peptide or mixture of peptides containing a lipidated cysteine residue, and a carrier. Accordingly, peptides of the present invention generally have at least one lipidated cysteine residue, and are also referred to herein as lipidated peptides. The inventive compositions may further contain non-lipidated peptides.

Peptides of the present invention may contain as many as 200 amino acid residues, or more, although in some embodiments, the peptide is shorter in length.

Peptides of the present invention may contain a lipidated cysteine residue at the C-terminus of the peptide. In some embodiments, the cysteine residue is located within about 5 residues of the C-terminus of the peptide.

In some embodiments, the peptide contains more than one cysteine residue. Of these residues, one, some or all may be lipidated. In such cases when two or more cysteine residues are present in a single peptide, the cysteine residues may be next to each other or one or more amino acid residues may separate each cysteine residue.

The lipid group may be bound to the cysteine residue by way of a chemical bond such as a thioether bond.

The lipidated peptides of the present invention may contain additional chemical modifications, such as in the N-terminal region. Chemical modification may be performed to the N-terminus or on other free amines in the peptide to enhance solubility, stability, or permeation of the lipidated peptide. Chemical modification techniques useful to enhance solubility, stability, or permeation of lipidated peptides are well known in the art. For example, techniques may include acetylation and/or BOC protection. Modifications may be made at or near the free amino group of the first amino acid in the peptide at the N-terminus in order to alter the chemical properties of the peptide. Other modifications that might be made are well known to those skilled in the art.

Lipids that may be useful for purposes of this invention are polyisoprenoids, including, for example, a geranyl group, farnesyl group, gernaylgeranyl group and phytyl group. More than one lipid may be used to modify cysteine residues in peptide. For example, if a peptide contains two or more cysteine residues, one cysteine residue may be lipidated with a phytyl group, while another cysteine residue may be lipidated with a farnesyl group.

In certain embodiments, inventive compositions containing peptides can be used effectively in topical applications to promote healthy epithelia or to treat epithelial-related disorders. These inventive compositions do not exhibit systemic effects when topically applied. Such inventive compositions are useful for, inter alia, their soothing, moisturizing and detergent properties, for treating cosmetic conditions and/or for generally promoting healthy skin. The compositions of the present invention may be usefully employed in cosmetic, cosmeceutical and general skincare compositions as well as in pharmaceutical compositions.

Below are certain definitions of terms used herein, many of which are understood by one skilled in the art.

The phrase “epithelia” or “epithelial” or “epithelial tissues” as used throughout the specification and claims is meant to include skin and mucosal membranes. Thus, the present invention offers compositions useful for treating a condition of the skin or a mucosal membrane, such as, but not limited to, that of a nose, a mouth, an eye, an ear, a vagina and a rectum.

In certain embodiments, compositions of the present invention include a pharmaceutical composition. As used herein, a “pharmaceutical composition” refers to a composition that is employed to prevent, reduce intensity, cure or otherwise treat a target condition or disease.

The terms “polypeptide” and “peptide” are used interchangeably herein. In certain embodiments, a “polypeptide” and “peptide” can refer to any sequence having 2 or more amino acids (e.g., 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, or more). In some embodiments, the number of amino acids is fewer than 1,000, 500, 400, 300, 200, 100, or so. In some embodiments, the term “dipeptide” refers to a “polypeptide” having 2 amino acids.

In certain embodiments, compositions of the present invention include a cosmetic composition, as defined herein.

### 1. Description of Exemplary Polypeptides

Peptides of the present invention may be generated from any known protein, peptide, or mixture of peptides suitable for topical application to keratinous tissue. Proteins known in the art and currently used in topical consumer products include soy protein, keratin, whey, casein, silk, wheat, rice, oat, and other natural sources of protein used in food or cosmetic products.

In addition, it is contemplated that naturally occurring enzymes within the cell signaling processes may be used to generate peptides of the present invention. For example, a peptide containing a CaaX motif or a lipidated cysteine may be applied topically, and then processed by endogenous enzymes to add a lipid in the case of the CaaX, and to remove the amino acids C-terminal to the cysteine in either case.

Table I depicts GENBANK® identifier and the C-terminal amino acid sequences of 113 of human proteins that exist in the human body in lipidated form. Included in Table I are sequences derived from G proteins. The four amino acid sequences at the C-terminal constitute the CaaX motif. The three amino acid residues after the cysteine residue of this motif can be removed after the cysteine residue at the C-terminus is lipidated. These modified peptides are thus representative examples of lipidated peptides of the present invention.

TABLE I
Exemplary Polypeptides

| GENBANK ® identifier | Sequence | SEQ ID NO. |
|---|---|---|
| gi\|9011186\|sp\|P51160\|PDE6C_HU | --GGAEKAAEDSGGGDDKKSKTCLML | 1 |
| gi\|544052\|sp\|P35913\|PDE6B_HUMA | --AAKKVGTEICNGGPAPKSSTCCIL | 2 |
| gi\|131852\|sp\|P10114\|RAP2A_HUMA | --IVRQMNYAAQPDKDDPCCSACNIQ | 3 |
| gi\|20981707\|sp\|P17964\|RAP3_HUM | --IVRQMNYAAQPNGDEGCCSACVIL | 4 |
| gi\|131855\|sp\|P10113\|RAPA_HUMAN | --LVRQINRKTPVEKKKPKKKSCLLL | 5 |
| gi\|131856\|sp\|P09526\|RAPB_HUMAN | --LVRQINRKTPVPGKARKKSSCQLL | 6 |
| gi\|1706474\|sp\|P31689\|DNJA1_HUM | --RHYNGEAYEDDEHHPRGGVQCQTS | 7 |
| gi\|27805462\|sp\|Q8WW22\|DNJA4_HU | --WRQHREAYEEDEDGPQAGVQCQTA | 8 |
| gi\|41018470\|sp\|Q8N5D0\|WDTC1_HU | --LSSGGAGASDDEDSSEGQVQCRPS | 9 |
| gi\|38258272\|sp\|Q9Y272\|RASD1_HU | --DLMYIREKASAGSQAKDKERCVIS | 10 |
| gi\|21362868\|sp\|Q96D21\|RHES_HUM | --DLKYIKAKVLREGQARERDKCTIQ | 11 |
| gi\|134080\|sp\|P17081\|RHOQ_HUMAN | --ILTPKKHTVKKRIGSRCINCCLIT | 12 |
| gi\|132542\|sp\|P01121\|RHOB_HUMAN | --TATRAALQKRYGSQNGCINCCKVL | 13 |
| gi\|1777755\|gb\|AAB40597.1\| | --EKYRPKMRLRFKDSNGHRNNCCIQ | 14 |
| gi\|1777757\|gb\|AAB40598.1\| | --LYLEKYRPKMRLRFRDTNGHCCVQ | 15 |
| gi\|14589856\|ref\|NP_116000.1\| | --EKYRPKQRLRFKDPHTHKTRCCVM | 16 |
| gi\|2833269\|sp\|Q15835\|RK_HUMAN | --MPDDMKGISGGSSSSSKSGMCLVS | 17 |
| gi\|21263659\|sp\|Q8WTQ7\|GRK7_HUM | --LNDPNRPTGCEEGNSSKSGVCLLL | 18 |
| gi\|2507282\|sp\|P17082\|RRA2_HUMA | --QECPPSPEPTRKEKDKKGCHCVIF | 19 |
| gi\|133486\|sp\|P10301\|RRAS_HUMAN | --EQELPPSPPSAPRKKGGGCPCVLL | 20 |
| gi\|145559475\|sp\|Q9UKC9\|FBXL2_H | --FAPVTPPTAVAGSGQRLCRCCVIL | 21 |
| gi\|38503141\|sp\|Q96IG2\|FXL20_HU | --FAPVTPPPSVGGSRQRFCRCCIIL | 22 |
| gi\|131869\|sp\|P01112\|RASH_HUMAN | --HKLRKLNPPDESGPGCMSCKCVLS | 23 |
| gi\|131883\|sp\|P01111\|RASN_HUMAN | --YRMKKLNSSDDGTQGCMGLPCVVM | 24 |
| gi\|5915765\|sp\|Q13072\|BAGE1_HUM | --MKEESPVVSWRLEPEDGTALCFIF | 25 |
| gi\|37537778\|sp\|Q86Y27\|BAGE5_HU | --MKEESPVVSWRLEPEDGTALCFIF | 26 |
| gi\|38604975\|sp\|Q96PZ7\|CSMD1_HU | --TNLKPTEAKAVRFDTTLNTVCTVV | 27 |
| gi\|38604740\|sp\|Q7Z407\|CSMD3_HU | --TNAKSVEGKAVRFDPNLNTVCTMV | 28 |
| gi\|417031\|sp\|P32455\|GBP1_HUMAN | --SRIMKNEIQDLQTKMRRRKACTIS | 29 |
| gi\|22653899\|sp\|O75781\|PALM_HUM | --PEATTSDPQDLDMKKHRCKCCSIM | 30 |
| gi\|2500182\|sp\|Q92730\|RND1_HUMA | --HLPSRSELISSTFKKEKAKSCSIM | 31 |
| gi\|1710230\|sp\|P52199\|RHOE_HUMA | --PSRPELSAVATDLRKDKAKSCTVM | 32 |
| gi\|21553323\|ref\|NP_660156.1\| | --LNIDGKRSGKQKRTDRVKGKCTLM | 33 |
| gi\|21703367\|ref\|NP_060064.2\| | --LQIDGKKSKQQKRKEKLKGKCVIM | 34 |
| gi\|13633743\|sp\|O95661\|DIRA3_HU | --LQEPEKKSQMPNTTEKLLDKCIIM | 35 |
| gi\|131875\|sp\|P01116\|RASK_HUMAN | --YRLKKISKEEKTPGCVKIKKCIIM | 36 |
| gi\|131879\|sp\|P01118\|RASL_HUMAN | --KHKEKMSKDGKKKKKKSKTKCVIM | 37 |
| gi\|1730223\|sp\|P50152\|GBGB_HUMA | --EDPLVKGIPEDKNPFKEKGSCVIS | 38 |
| gi\|585181\|sp\|Q08447\|GBG1_HUMAN | --DPLVKGIPEDKNPFKELKGGCVIS | 39 |
| gi\|3023844\|sp\|O14610\|GBGT2_HUM | --NDPFLKGIPEDKNPFKEKGGCLIS | 40 |
| gi\|20138402\|sp\|Q9P2W3\|GBG13_HU | --KDPFLNPDLMKNNPWVEKGKCTIL | 41 |
| gi\|12229817\|sp\|Q9UBI6\|GBG12_HU | --SDPLLIGIPTSENPFKDKKTCIIL | 42 |
| gi\|6016106\|sp\|O60262\|GBG7_HUMA | --NDPLLVGVPASENPFKDKKPCIIL | 43 |
| gi\|12229849\|sp\|Q9Y3K8\|GBG5L_HU | --HDPLLTGVSSSTNPFRPQKVCSFL | 44 |
| gi\|232147\|sp\|P30670\|GBG5_HUMAN | --HDPLLTGVSSSTNPFRPQKVCSFL | 45 |
| gi\|32699499\|sp\|P59768\|GBG2_HUM | --DPLLTPVPASENPFREKKFFCAIL | 46 |
| gi\|232146\|sp\|P29798\|GBG3_HUMAN | --DPLITPVPTSENPFREKKFFCALL | 47 |
| gi\|1730219\|sp\|P50150\|GBG4_HUMA | --DPLIIPVPASENPFREKKFFCTIL | 48 |
| gi\|12232629\|sp\|Q9UK08\|GBG8_HUM | --DPLVTPVPAAENPFRDKRLFCVLL | 49 |
| gi\|1730222\|sp\|P50151\|GBG10_HUM | --KDALLVGVPAGSNPFREPRSCALL | 50 |
| gi\|22256976\|sp\|Q96I34\|PP16A_HU | --PPLLKLTAPAVEAPVERRPCCLLM | 51 |
| gi\|22256977\|sp\|Q96T49\|PP16B_HU | --PPLLKFKAPIEEMEEKVHGCCRIS | 52 |
| gi\|125953\|sp\|P20700\|LMNB1_HUMA | --VVVEEELFHQQGTPRASNRSCAIM | 53 |
| gi\|23503078\|sp\|Q03252\|LMNB2_HU | --EFGEEDLFHQQGDPRTTSRGCYVM | 54 |
| gi\|131807\|sp\|P15154\|RAC1_HUMAN | --FDEAIRAVLCPPPVKKRKRKCLLL | 55 |
| gi\|13633387\|sp\|O95916\|RAC4_HUM | --FDEAIRAVLCPPPVKKRKRKCLQL | 56 |
| gi\|46397673\|sp\|P60763\|RAC3_HUM | --FDEAIRAVLCPPPVKKPGKKCTVF | 57 |
| gi\|131806\|sp\|P15153\|RAC2_HUMAN | --FDEAIRAVLCPQPTRQQKRACSLL | 58 |
| gi\|464611\|sp\|P35238\|RHOG_HUMAN | --VFAEAVRAVLNPTPIKRGRSCILL | 59 |
| gi\|46397381\|sp\|P60953\|CDC42_HU | --VFDEAILAALEPPETQPKRKCCIF | 60 |
| gi\|132540\|sp\|P06749\|RHOA_HUMAN | --VFEMATRAALQARRGKKKSGCLVL | 61 |
| gi\|132543\|sp\|P08134\|RHOC_HUMAN | --VFEMATRAGLQVRKNKRRRGCPIL | 62 |
| gi\|1170685\|sp\|P46019\|KPB2_HUMA | --MTYLTRAVASYLQELLPNSGCQMQ | 63 |
| gi\|1170683\|sp\|P46020\|KPB1_HUMA | --MTYLSKAAATYVQEFLPHSICAMQ | 64 |
| gi\|2507301\|sp\|P52198\|RND2_HUMA | --LSGRPDRGNEGEIHKDRAKSCNLM | 65 |
| gi\|1706299\|sp\|P51398\|RT29_HUMA | --EGKKELLFLSNANPSLLERHCAYL | 66 |
| gi\|2851392\|sp\|P16499\|PDE6A_HUM | --AAGNQPGGNPSPGGATTSKSCCIQ | 67 |
| gi\|729723\|sp\|P40855\|PEX19_HUMA | --LNFDLDALNLSGPPGASGEQCLIM | 68 |
| gi\|125962\|sp\|P02545\|LMNA_HUMAN | --LVTRSYLLGNSSPRTQSPQNCSIM | 69 |
| gi\|116013\|sp\|P01730\|CD4_HUMAN | --IKRLLSEKKTCQCPHRFQKTCSPI | 70 |
| gi\|8925284\|gb\|AAF81404.1\|AF187 | --RISKEIQRQQALQSQNSSTICSVS | 71 |
| gi\|13633711\|sp\|Q9HBH0\|RHOF_HUM | --AAKVALSALKKAQRQKKRRLCLLL | 72 |
| gi\|59803021\|sp\|P32019\|I5P2_HUMA | --HQKLDMTEKKKAQEFIHQFLCNPL | 73 |
| gi\|11135165\|sp\|O95858\|TSN15_HU | --GLLGPGAKPSVEAAGTGCCLCYPN | 74 |
| gi\|1172500\|sp\|P43119\|PI2R_HUMA | --TQQSSGSAVGTSSKAEASVACSLC | 75 |
| gi\|3024572\|sp\|Q92737\|RSLAA_HUM | --VRARPAHPALRLQGALHPARCSLM | 76 |
| gi\|46200326\|emb\|CAE51163.1\| | --NQQFPWVDPGPRPPRLPLLECTPQ | 77 |
| gi\|24418646\|sp\|Q9H4E5\|RHOJ_HUM | --ILTIFHPKKKKKRCSEGHSCCSII | 78 |
| gi\|1345731\|sp\|P49454\|CENPF_HUM | --KRGRLVPSPKAGLESKGSENCKVQ | 79 |
| gi\|6919957\|sp\|Q15382\|RHEB_HUMA | --RRIILEAEKMDGAASQGKSSCSVM | 80 |
| gi\|9087144\|sp\|O15255\|CXX1_HUMA | --PPRDLSPSARPISSPPPETSCVLA | 81 |
| gi\|399227\|sp\|Q02224\|CENPE_HUMA | --ESVDSQPGPWHASSGKDVPECKTQ | 82 |
| gi\|1705945\|sp\|P09543\|CN37_HUMA | --YGKGKPVPTQGSRKGGALQSCTII | 83 |
| gi\|67476964\|sp\|P48448\|AL3B2_HU | --YPPYTDWNQQLLRWGMGSQSCTLL | 84 |
| gi\|25009267\|sp\|Q9H078\|CLPB_HUM | --DKDSKTRRLDIRAPLHPEKVCNTI | 85 |
| gi\|22550104\|ref\|NP_115971.2\| | --KKMADTSSMDEDFESDYKKYCVLQ | 86 |
| gi\|1168340\|sp\|P45381\|ACY2_HUMA | --EKKEAFAKTTKLTLNAKSIRCCLH | 87 |
| gi\|6226045\|sp\|O14807\|RASM_HUMA | --KKKKKTKWRGDRATGTHKLQCVIL | 88 |
| gi\|1169868\|sp\|P32456\|GBP2_HUMA | --KRLQKDIWDIQMRSKSLEPICNIL | 89 |
| gi\|131834\|sp\|P11233\|RALA_HUMAN | --SKEKNGKKKRKSLAKRIRERCCIL | 90 |
| gi\|25304072\|gb\|AAH40076.1\| | --SKSRTPDKMKNLSKSWWKKYCCFV | 91 |
| gi\|26006843\|sp\|O94955\|RHBT3_HU | --NMYLKQLAEYRKYIHSRKCRCLVM | 92 |
| gi\|462567\|sp\|P34949\|MANA_HUMAN | --ANESVSLKLTEPKDLLIFRACCLL | 93 |
| gi\|33860221\|sp\|Q9Y6Q2\|STON1_HU | --VEIEKKWIKIDGEDPDKIGDCITQ | 94 |
| gi\|1169285\|sp\|P43353\|AL3B1_HUM | --PQSPRRLRMLLVAMEAQGCSCTLL | 95 |
| gi\|2500200\|sp\|Q15669\|RHOH_HUMA | --RTAVNQARRRNRRRLFSINECKIF | 96 |
| gi\|17433280\|sp\|O95236\|APOL3_HU | --AQELEENLMELTQIYQRLNPCHTH | 97 |
| gi\|2498243\|sp\|Q13286\|CLN3_HUMA | --SDTLGISLSGLLALPLHDFLCQLS | 98 |
| gi\|2499582\|sp\|Q93100\|KPBB_HUMA | --TKAVMNLLLEGEVKPNNDDPCLIS | 99 |
| gi\|37999757\|sp\|Q96PP8\|GBP5_HUM | --NRSLLSELQHAQRTVNNDDPCVLL | 100 |
| gi\|13123995\|sp\|Q9Y2C3\|B3GT5_HU | --IKPRTLLDYWQALENSRGEDCPPV | 101 |
| gi\|3122245\|sp\|Q14642\|I5P1_HUMA | --FLAFRIMPGAGKPHAHVHKCCVVQ | 102 |
| gi\|22096354\|sp\|O43900\|LMO6_HUM | --SLSLPRDSRAGMPRQARDKNCIVA | 103 |
| gi\|127846\|sp\|P00387\|NB5R3_HUMA | --PMIQYACLPNLDHVGHPTERCFVF | 104 |
| gi\|77416872\|sp\|Q96M98\|PACRG_HU | --RYGGENAFINIKYVVPTYESCLLN | 105 |
| gi\|12643622\|sp\|O60635\|TSN1_HUM | --VAAGIGGLELAAMIVSMYLYCNLQ | 106 |
| gi\|23396579\|sp\|P59022\|DSC10_HU | --PFCAEPCSRVTVCHLQAVPVCMPV | 107 |
| gi\|24211596\|sp\|Q9BUV8\|CT024_HU | --LFMVCVADSFTTGHLDHLLHCHPL | 108 |
| gi\|13123990\|sp\|Q9UBV7\|B4GT7_HU | --GAPCTVLNIMLDCDKTATPWCTFS | 109 |
| gi\|9087132\|sp\|Q16589\|CCNG2_HUM | --SPPSDQECTFFFNFKVAQTLCFPS | 110 |
| gi\|37999851\|sp\|Q8WVZ1\|ZDH19_HU | --AAASWMRLASASCRAKPWAVCFPS | 111 |
| gi\|3024539\|sp\|O00212\|RHOD_HUMA | --AEVALSSRGRNFWRRITQGFCVVT | 112 |
| gi\|12585259\|sp\|Q9NQX7\|ITM2C_HU | --AKNCNAIRHFENTFVVETLICGVV | 113 |

When choosing a peptide (or polypeptide) or combination of peptides (or polypeptides) from Table I for use in the present invention, one skilled in the art may look at the conservation of the upstream amino acid residues. While there is little if any homology or sequence similarity of amino acid sequences upstream of (or N-terminal with respect to) the C-terminal cysteine residue, there is some conservation of the amino acid residue immediately upstream from the C-terminal cysteine residue. Table II shows the conservation of the amino acid residue immediately upstream of the cysteine residue of the CaaX motif. It is believed that a more highly conserved amino acid residue will lead to greater activity of the specific peptide for cell signaling binding sites. For example, serine appears to be the most prevalent amino acid residue upstream from the cysteine residue of the CaaX motif and therefore peptides with a serine residue immediately upstream of a cysteine residue in the C-terminal region may have more activity with respect to cell regulation and signaling proteins. Without being bound by any particular theory or mechanism of action, peptides of the present invention may act to mimic or match sequences such as those in Table I and thus be more active than single amino acid mimics like N-acetyl-5-farnesyl-cysteine (AFC).

TABLE II
*Amino Acid upstream to the Cys of the CaaX motif

| Amino Acid | Total | % of CaaX | ×100 |
|---|---|---|---|
| S | 16 | 0.14159204 | 14.15920 |
| K | 12 | 0.10619469 | 10.61947 |
| C | 9 | 0.07964602 | 7.964602 |
| V | 8 | 0.07079646 | 7.079646 |
| R | 7 | 0.06194690 | 6.194690 |
| L | 7 | 0.06194690 | 6.194690 |
| A | 6 | 0.05309735 | 5.309735 |
| P | 6 | 0.05309735 | 5.309735 |
| G | 6 | 0.05309735 | 5.309735 |
| Q | 5 | 0.04424779 | 4.424779 |
| F | 5 | 0.04424779 | 4.424779 |
| T | 4 | 0.03539823 | 3.539823 |
| N | 4 | 0.03539823 | 3.539823 |
| H | 4 | 0.03539823 | 3.539823 |
| I | 4 | 0.03539823 | 3.539823 |
| E | 3 | 0.02654867 | 2.654867 |
| Y | 3 | 0.02654867 | 2.654867 |
| D | 2 | 0.01769912 | 1.769912 |
| M | 1 | 0.00884956 | 0.884956 |
| W | 1 | 0.00884956 | 0.884956 |
| Total | 113 | | |

Known methods in the art may be used to prepare a peptide of desirable length for purposes of the present invention. By way of example, a natural protein may be partially hydrolyzed to achieve a mixture of peptides of about 2-200 amino acid residues, or more.

Not all naturally occurring peptides contain a cysteine residue. Nonetheless, such peptides can be modified to include a lipidated cysteine at any desired position in the peptide sequence, e.g., within 5 residues of the C-terminus, either by introducing the cysteine residue and then adding the lipid group, or by directly introducing a lipidated cysteine residue into to peptide sequence. For example, farnesyl cysteine (FC) may be coupled to a peptide at the N or C-terminus. See Example 1.

In addition to peptides derived from natural protein sources, e.g., soy protein, keratin, whey, casein, proteins and the like, a synthetic peptide may be used as a starting material to synthesize peptides useful for purposes of the present invention. Like peptides derived from natural protein, synthetic peptides may be modified to include a cysteine residue.

When a peptide of the present invention contains two or more cysteine residues, and subsequent lipidation of the cysteine residues is desired, it is understood that by using conventional laboratory protocols, all cysteine residues present on the peptide may become lipid modified, i.e., lipidated with a single lipid.

Lipidated peptides of the present invention may be generated either by chemical modification or enriching for lipid modified peptides.

Chemical modification may be used to increase the content of lipid-modified cysteine residues within a desired peptide. In one embodiment, a lipid group and peptide containing a cysteine exist separately. The peptide may be chemically modified and thus, lipidated at the cysteine residue. By way of example, a free cysteine residue or peptide containing a cysteine residue may be reacted with an activated lipid such as farnesyl-bromide under suitable conditions such that free cysteine residues become lipidated. See Example 2.

In another embodiment, a cysteine residue may be lipidated and then the lipidated cysteine residue may be used to modify a peptide so that the lipidated cysteine residue is at the C-terminus. A lipid-modified cysteine, such as farnesyl-cysteine, could be coupled to the C-terminus of naturally-derived peptides by protecting the amino groups in the peptide, such as with Fmoc or BOC, and then activating the peptide C-terminus with an activating group and reacting them together. The lipid-modified cysteine could also be coupled to a solid-phase support to enhance the process. See Example 3.

In one such method, attachment of lipid moieties such as a farnesyl to a cysteine residue is performed by reacting the sulfhydryl group of cysteine with farnesyl bromide under basic conditions to attach the farnesyl group. In another method, lipidated peptides are made by using a lipid mercaptan to displace the bromide of a bromoalanine containing peptide. For example, farnesyl mercaptan can be synthesized according to the methods described in U.S. Pat. No. 3,429,970 or Gilbert, et al., J. Am Chem. Soc. 114:3966-3913 (1992).

It is also known that lipidated peptides of the present invention may exist naturally. In such instances, it is desirable to enrich for lipidated peptides, in which case they are extracted or isolated from their natural environment. This typically involves taking a natural source of lipidated peptides, i.e., hydrolyzed protein, and using a chromatographic or chemical separation to partially purify the mixture and thus increase the percentage of the peptides in the product that contain a lipid modified cysteine residue. Thus, a higher percentage of lipid modified peptides are present in the product than in the original protein source.

Alternatively, peptides of the present invention that have been chemically modified and thus lipidated may also be enriched. In this regard, a higher percentage of lipidated peptides or lipid modified cysteine residues will result.

Methods of enriching for lipidated peptides or lipidated cysteine residues are well known in the art. By way of example, enriching may include use of antibodies or aptamers that specifically recognize lipidated peptides. See Gilbert, et al., Bioorganic and Medicinal Chemistry. 5(6):1115-22 (1997).

In addition, column chromatography, solid-phase, or liquid extraction whose selectivity is based on the hydrophobic nature of the lipid may also be used to enrich for lipid modification. In this case a resin with general or specific affinity for lipids or lipid modified Cysteine residues may be used, i.e., C4 resin, a resin coupled to a hydrophobic dye molecule, serum albumin coupled to a solid support, or a resin with a custom functional group or binding site. Other standard methods of solid phase synthesis are described in Lumbierres, et al., 11(24):7405-15 (2005); Pachamutha, et al., J. Org. Chem. 70(9):3720-3 (2005) and Douat et al., J. Pept. Sci. 8(11):601-14 (2002), and Dolence et al., J. Comb. Chem. 2(5):522-36 (2000), all of which are herein incorporated herein by reference. See Example 3.

### 2. Modulation of the Natural Signaling Process

Peptides of the present invention may be substrates for isoprenylcysteine carboxyl methyltransferase (ICMT). ICMT is a gene that encodes one of three enzymes that post-translationally modify isoprenylated C-terminal cysteine residues in certain proteins and target those proteins to the cell membrane. This enzyme localizes to the endoplasmic reticulum.

Similar to AFC, and again without intending to be bound by a particular theory of operation, lipidated peptides of the present invention may compete with cellular targets for modification by ICMT. In addition, lipidated peptides of the present invention may also compete with G-proteins or other peptides modified with farnesyl, geranylgeranyl, or palmitoyl for sites of interaction.

For example, yeast a-factor is a farnesylated peptide, and is methylated by the same enzyme in the same fashion as the G-proteins. See Hrycyna, et al., EMBO J. 10(7):1699-709 (1991). Synthetic farnesylated or geranylgeranylated peptides can be methylated by mammalian ICMT. See Stephenson and Clarke J. Biol Chem. 265(27):16248-54 (1990), Giner and Rando, Biochemistry 33(50):15116-23 (1994) which are all herein incorporated herein by reference.

In addition, a subset of modified peptides may have a higher affinity for their cellular targets and thus may have superior potency to AFC in biochemical, and cellular assays and thus may be useful in the sprit of the present invention. Without being bound by any particular theory, applicants believe this to be the case because the lipidated peptides of the present invention have amino acids in addition to the modified cysteine residue in AFC.

### 3. Topical Preparations

The peptides of the present invention may be used with a carrier suitable for topical administration and thus are said to be cosmetically, pharmaceutically or dermatologically acceptable. Carriers are non-toxic to keratinous tissue such as skin and are generally inert, although the carrier can also provide a benefit.

Topical preparations may be uniformly lipid modified. In other words, all lipidated peptides included within a topical preparation may be modified with a single lipid. On the other hand, not every single peptide in the composition is necessarily lipidated. For example, in a partially hydrolyzed protein extract, not all peptides will contain a cysteine residue. Hence, if treated with farnesyl-bromide, only the fraction containing a cysteine residue will be lipidated (see Example 2). Thus, the term “uniformly lipid modified” also refers to all lipidated peptides within a topical preparation that are in a well-defined and consistent distribution. For example, the lipidated peptides contained in a topical preparation may be modified with two or more lipids and be in a well-defined and consistent distribution so as to make the topical preparation uniformly lipid modified.

The term “topical” refers to administration of inventive compositions at, or immediately beneath, the point of application.

Topical applications referred to herein are for application onto one or more surfaces(s) including keratinous tissue, i.e., “topically applying.” Topical application or “topically applying” may involve direct application to the area of the desired substrate. The topical preparation and/or composition may be applied by pouring, dropping, or spraying, if a liquid; rubbing on, if an ointment, lotion, cream, gel, or the like; dusting, if a powder; spraying, if a liquid or aerosol composition; or by any other appropriate means.

In another embodiment, the topical preparation and/or composition. As used herein, a “cosmetic composition” refers to a composition that is intended to be rubbed, poured, sprinkled, or sprayed on, introduced into, or otherwise applied to a substrate or any part thereof for cleansing, beautifying, promoting attractiveness, or altering the appearance, or an article intended for use as a component of any such article.

Lipidated peptides are present in topical compositions in concentrations generally ranging from 0.01% to about 10% w/v of the topical composition. In some embodiments, the amount of lipidated peptides may exceed 10% w/v. More typically, however, lipidated peptides are present in topical compositions between 0.1% and 5% w/v. Such amounts are considered “effective” for purposes of the uses of the compositions as described herein.

Choice of carrier element(s) depends upon the type of formulation envisioned. The topical preparations may be applied locally to the skin, hair, scalp or mucosa and may be in any form including solutions, oils, creams (e.g., moisturizers), ointments, emulsions (e.g., shampoos), suspensions, gels, lotions, milks, cleansers, sprays, transdermal patches and the like. See Example 4.

In another embodiment, a polyisoprenyl-protein inhibitor compound, carrier and, optionally, additional active ingredients are formed into a composition.

A solution may be prepared by mixing a solute or dissolved substance (such as a lipidated peptide and, optionally, one or more active ingredient(s)) uniformly throughout a solvent carrier such as water or organic solvents, such as lower alcohols (e.g., lower alkanols such as ethanol and isopropanol) and acetone. A basic emulsion contains at least three components, the two immiscible liquid carriers and the emulsifying agent as well as the lipidated peptide. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Gels differ from emulsions in one respect in that they do not contain emulsifiers.

In yet, another embodiment, the lipidated peptides may be mixed with a gel suspension, (a semi-solid carrier) or solid carrier to form a paste, powder, ointment, cream, lotion, hydrogel or the like.

For example, ointments may be prepared which are in gel-suspension form. These are semi-solid preparations intended for external application to the epithelium. Generally, ointment bases are categorized into hydrocarbon bases (oleaginous), which may use white petroleum as a base; adsorption bases (anhydrous), which might use hydrophilic petroleum or anhydrous lanolin; emulsion bases (water and oil type); emulsion bases (oil and water type); and water soluble bases, which often use polyethylene glycol as an ointment base.

Additional topical preparations using lipidated peptides and carriers can be readily prepared using technology which is known in the art such as described in Remington's Pharmaceutical Sciences, 18th or 19th editions, published by the Mack Publishing Company of Easton, Pa.

Accordingly, non-limiting representative examples of carriers include moisturizing agents or humectants, pH adjusting agents, a deodorant agent, fragrances, hair conditioning agents, chelating agents, preservatives, emulsifiers or surfactants, thickeners, solubilizing agents, penetration enhancers, anti-irritants and colorants.

As used herein a “moisturizing agent” is a substance that adds or restores moisture to the skin. Representative examples of moisturizing or humectant agents that are usable in the present invention include, without limitation, guanidine, glycolic acid and glycolate salts (e.g., ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

As is widely recognized in the art, since the pH of the skin is 5.5, topical preparations for skin application, to avoid irritation, should preferably have a pH value of between 4.0 and 7.0, preferably between 5.0 and 6.0, most preferably about 5.5 or substantially 5.5. Accordingly, a pH adjusting composition is typically added to bring the pH of the composition to the desired value. The topical preparation therefore preferably are formulated to have a pH value that ranges between about 4.0 and about 7.0, more preferably between about 5.0 and about 6.0.

Suitable pH adjusting agents include, for example, but are not limited to, one or more adipic acids, glycines, citric acids, calcium hydroxides, magnesium aluminometasilicates, buffers or any combinations thereof.

As used herein “deodorant agent” refers to a substance for inhibiting or masking perspiration or other bodily odors. Representative examples of deodorant agents that may be used include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmlthyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g., as the alkali metal carbonates and bicarbonates, ammonium and tetraalkylammonium carbonates and bicarbonates, especially the sodium and potassium salts, or any combination of the above. Antiperspirant agents can be incorporated in the compositions of the present invention either in a solubilized or a particulate form and include, for example, aluminum or zirconium astringent salts or complexes.

As used herein “fragrance” refers to a substance having an aroma. Suitable fragrances include, but are not limited to, eucalyptus oil, camphor synthetic, peppermint oil, clove oil, lavender, chamomile and the like.

Suitable hair conditioning agents that may be used include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

Chelating agents may be optionally added to the topical preparation so as to enhance the preservative or preservative system. Preferred chelating agents are mild agents, such as, for example, ethylenediaminetetraacetic acid (EDTA), EDTA derivatives, or any combination thereof.

Suitable preservatives for use may include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Emulsifiers (also known as surfactants), as used herein promote the formation and stabilization of an emulsion. Suitable emulsifiers may be natural materials, finely divided solids, or synthetic materials. Natural emulsifying agents may be derived from either animal or vegetable sources. Those from animal sources may include gelatin, egg yolk, casein, wool fat, or cholesterol. Those from vegetable sources may include acacia, tragacanth, chondrus, or pectin. Vegetable sources specifically from cellulose derivatives include methyl cellulose and carboxymethyl cellulose to increase the viscosity. Finely divided emulsifiers may include bentonite, magnesium hydroxide, aluminum hydroxide, or magnesium trisylicate. Synthetic agents are anionic, cationic, zwitterionic or nonionic. Examples of emulsifiers employed in topical compositions include sodium lauryl sulfate, benzalkonium chloride, polyethylene glycol 400 monostearate, and combinations thereof.

“Thickeners” (also known as gelling agents) as used herein refer to agents that make a topical preparation denser or more viscous in consistency. Thickeners are water-based or oil-based. Suitable thickeners that may be used in the context of the present invention include, for example, non-ionic water-soluble polymers such as hydroxyethylcellulose (commercially available under the Trademark Natrosol™ 250 or 350), cationic water-soluble polymers such as Polyquat 37 (commercially available under the Trademark Synthalen™ CN), fatty alcohols, fatty acids, anionic polymers, and their alkali salts and mixtures thereof.

As used herein “solubilizing agents” are those substances that enable solutes to dissolve. Representative examples of solubilizing agents that may be used in the context herein include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN™ and spans, e.g., TWEEN 80™. Other solubilizers that are usable for topical preparations may be, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

A “penetration enhancer” is an agent known to accelerate the delivery of a substance through the skin. Suitable penetration enhancers may include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacyclo-heptan-2-one (available under the trademark Azone™ from Whitby Research Incorporated, Richmond, Va.), alcohols, and the like. The permeation enhancer may also be a vegetable oil. Such oils may include, for example, safflower oil, cottonseed oil and corn oil.

Additional thickeners, penetration enhancers and other carrier materials or adjuvants may generally be found in Remington's Pharmaceutical Sciences, 18th or 19th editions, published by the Mack Publishing Company of Easton, Pa. which is herein incorporated herein by reference.

As used herein, an “anti-irritant” is an agent that prevents or reduces soreness, roughness, or inflammation of a bodily part. Suitable anti-irritants that may be used include, for example, but are not limited to, steroidal and non steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

The presently known anti-irritants can be divided into water-soluble anti-irritants and water-insoluble anti-irritants. Representative examples of such compositions are described, for example, in U.S. Pat. No. 5,482,710 which is herein incorporated herein by reference.

Colorants may include pigments or dyes or a combination thereof. Preferred pigments may include, but are not limited to, iron oxides, and titanium oxides. Suitable dyes include FD&C approved colorants, D&C approved colorants, and those approved for use in Europe and Japan. See Marmion, D. M., Handbook of US Colorants for Food, Drugs, Cosmetics, and Medical Devices, 3rd ed, 1991 herein incorporated herein by reference.

In certain embodiments, a pharmaceutically acceptable carrier is included in compositions of the present invention. As used herein, “a pharmaceutically acceptable carrier” is any substantially non-toxic carrier conventionally useable for topical administration of pharmaceuticals in which the peptide will remain stable and bioavailable when applied directly to skin or mucosal surfaces.

In certain embodiments, compositions of the present invention include a cosmetically acceptable carrier. As used herein, the phrase “cosmetically acceptable carrier” refers to a substantially non-toxic carrier, conventionally useable for the topical administration of cosmetics, with which polypeptides of the present invention will remain stable and bioavailable. It will be understood by one skilled in the art that cosmetically acceptable carriers and pharmaceutically acceptable carriers are similar, if not often identical, in nature.

Other suitable carriers of the present invention may include pharmaceutically acceptable carriers. For example, water, petroleum jelly (VASELINE®), mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, carriers described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated herein by reference, and the like. Also included are other carriers as described herein.

The carrier of the composition of the present invention may provide a sustained release or delayed release effect. The carrier can be any material capable of sustained or delayed release of lipidated peptides to provide a more efficient administration resulting in less frequent and/or decreased dosage of lipidated peptides, ease of handling, and extended or delayed effects on epithelial-related conditions. Non-limiting examples of such carriers may include liposomes, microsponges, microspheres, or microcapsules of natural and synthetic polymers and the like. Liposomes which may enhance the localized delivery of lipidated peptides within skin layers may be formed from a variety of phospholipids, such as cholesterol, stearylamines or phosphatidylcholines.

In some embodiments, the carrier of the composition of the present invention contains a film-forming polymer. Examples of topical carriers with film-forming polymers include sunscreens (U.S. Pat. No. 5,653,965), topical disinfectants (U.S. Pat. No. 7,323,163), and barrier compositions (U.S. Pat. No. 6,210,688). Other examples include carriers containing commercial film-forming polymers such as dehydroxanthan gum, Eastman AQ 38S, Dermacryl AQF®, Dermacryl 79®, Dermacryl LT®, Dermacryl C®, Aquamere, Avalure™ and the like. Such a film-forming polymer may impart water-resistant characteristics, or delayed-release characteristics.

In some embodiments, the topical preparation may be incorporated into a carrier which is suitable for administration to the oral mucosa, e.g., a mouthwash, rinse, oral spray, suspension, dental gel, and the like. Typical oral carriers known in the art may be used in the present invention. The pH value of the oral vehicle is generally from about 4 to about 7, and preferably from about 5 to about 6.5. The oral topical preparation may further contain conventional additives normally employed in those products such as a fluorine providing compound and a sweetening agent, providing the additives do not interfere with the therapeutic or cosmetically beneficial properties of the inventive compositions.

Fluorine providing compounds may be fully or slightly water soluble and are characterized by their ability to release fluoride ions or fluoride containing ions in water and by their lack of reaction with other components in the composition. Typical fluorine providing compounds are inorganic fluoride salts such as water-soluble alkali metal, alkaline earth metal, and heavy metal salts, for example, sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminum mono- and di-fluorophosphates and fluorinated sodium calcium pyrophosphate. Alkali metal fluorides, tin fluoride and monofluorophosphates, such as sodium and stannous fluoride, sodium monofluorophosphate and mixtures thereof, are preferred.

The amount of fluorine providing compound present may be dependent upon the type of fluorine providing compound employed, the solubility of the fluorine compound, and the nature of the final oral topical preparation. The amount of fluorine providing compound used must be a nontoxic amount. In general, the fluorine providing compound when used will be present in an amount up to about 1%, preferably from about 0.001% to about 0.1%, and most preferably from about 0.001% to about 0.05%, by weight of the oral topical preparation.

When sweetening agents (sweeteners) are used, those sweeteners well known in the art, including both natural and artificial sweeteners, may be employed. The sweetening agent used may be selected from a wide range of materials including, but not limited to, water-soluble sweetening agents, water-soluble artificial sweetening agents, water-soluble sweetening agents derived from naturally occurring water-soluble sweetening agents, dipeptide based sweetening agents, and protein based sweetening agents, including mixtures thereof.

The topical composition may further include one or more additional compatible active ingredients which are aimed at providing the topical preparation with another pharmaceutical, cosmeceutical or cosmetic effect, in addition to that provided by a lipidated peptide. “Compatible” as used herein means that the components of such a composition are capable of being combined with each other in a manner such that there is no interaction that would substantially reduce the efficacy of the topical preparation under ordinary conditions of use.

As used herein, the phrase “additional active ingredient” refers to an agent, other than the lipidated peptide, that exerts a pharmacological, dermatological or any other beneficial activity such as a cosmetic effect. It is to be understood that “other beneficial activity” may be one that is only perceived as such by the subject using the topical preparation described herein.

Topical preparations containing lipidated peptides, which further include one or more additional active ingredients, can therefore be further efficiently used, in addition to their use as a treatment for an epithelial-related condition, or simply as a cosmetic.

Additional active ingredients, without limitation, may include one or more, in any combination, of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a vitamin, a hormone, an anti-dandruff agent, an anti-wrinkle agent, an anti-skin atrophy agent, a sclerosing agent, a cleansing agent, a caustic agent and a hypo-pigmenting agent.

A “protective” is any agent that isolates the exposed surface of the skin or other membrane from harmful or annoying stimuli. Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a desiccant. The absorption of skin moisture decreases friction and also discourages certain bacterial growth. Some of the materials used as protective adsorbents include bentonite, insoluble salts of bismuth, boric acid, calcium carbonate, (precipitated), cellulose, corn starch, magnesium stearate, talc, titanium dioxide, zinc oxide, and zinc stearate.

Protectives also can be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples may include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

An example of a protective that may be included is demulcents. Demulcents are protective agents employed primarily to alleviate irritation, particularly mucous membranes or abraded tissues. They often are applied to the surface in a viscid, sticky preparation that covers the area readily and may be medicated. A number of chemical substances possess demulcent properties. These substances may include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

“Emollients” are generally bland, fatty or oleaginous materials which can be applied locally, particularly to the skin. Emollients increase the tissue moisture content, thereby rendering the skin softer and more pliable. Increased moisture content in the skin can be achieved by preventing water loss with an occlusive water-immiscible barrier, by increasing the water-holding capacity in the skin with humectants, or by altering the desquamation of the outermost skin layer, the stratum corneum. Useful emollients include lanolin, spermaceti, mineral oil, paraffin, petrolatum, white ointment, white petroleum, yellow ointment. Also included are vegetable oils, waxes, cetyl alcohol, glycerin, hydrophilic petrolatum, isopropyl myristate, myristyl alcohol, and oleyl alcohol.

“Astringents” are locally applied, generally protein precipitants, that have such a low cell penetrability that the action essentially is limited to the cell surface and interstitial spaces. The astringent action is accompanied by contraction and wrinkling of the tissue and by blanching. Astringents are used therapeutically to arrest hemorrhage by coagulating the blood, to promote healing, to toughen the skin or to decrease sweating. The principal components of astringents are salts of aluminum, zinc, manganese, iron or bismuth.

An “irritant” is a material that may act locally on the skin to induce, based on irritant concentration, hyperemia, inflammation, and desiccation. Irritant agents include, but are not limited to, alcohol, aromatic ammonia spirits, benzoin tincture, camphor capsicum, and coal tar extracts. Preferably, the irritant is a rubefacient. As used herein “rubefacients” are agents that may induce hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

Representative examples of sun screening agents usable in context of the present invention may include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, alpha-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Representative examples of sunless tanning agents usable in the present invention may include, without limitation, dihydroxyacetone, glyceraldehyde, indoles and their derivatives. The sunless tanning agents can be used in combination with the sunscreen agents.

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-fungal agent” as used herein means any of a group of chemical substances having the capacity to inhibit the growth of or to destroy fungi. Anti-fungal agents include but are not limited to Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, PyrroInitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferon-alpha, Interferon-beta, Interferon-gamma, Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation include pyrimethamine (DARAPRIM®) sulfadiazine, and Leucovorin.

Suitable anti-acne agents may include, without limitation, keratolytics, such as salicylic acid, sulfur, glycolic, pyruvic acid, resorcinol, and N-acetylcysteine; and retinoids such as retinoic acid and its derivatives (e.g., cis and trans, esters) and retinol.

“Anesthetic agents” refers to agents that resulting in a reduction or loss of sensation. Non-limiting examples of anesthetic drugs that are suitable may include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “pharmaceutically acceptable salt(s)” as used herein may include but is not limited to salts of acidic or basic groups that may be present in compounds of the present invention. Compounds that are basic in nature may be capable of forming a wide variety of salts with various inorganic and organic acids. Such non-toxic salts, i.e., salts containing pharmacologically acceptable anions, may include but are not limited to hydrochloride, hydrobromide, hydroiodide, nitrate, sulfate, bisulfate, phosphate, acid phosphate, isonicotinate, acetate, lactate, salicylate, citrate, acid citrate, tartrate, oxalate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, ptoluenesulfonate and pamoate (i.e., 1,1′-methylene-bis-(2-hydroxy-3-naphthoate)) salts. Compounds of the present invention that may include an amino group also may form pharmaceutically acceptable salts with various amino acids, in addition to the acids mentioned above. Compounds that may be acidic in nature are capable of forming base salts with various pharmacologically acceptable cations. Examples of such salts may include alkali metal or alkaline earth metal salts and, particularly, calcium, magnesium, sodium lithium, zinc, potassium, and iron salts. Other such salts may include pharmaceutically acceptable organic bases such as ammonia, arginine, benethamine, benzathine, deanol, diethanolamine, diethylamine, -2-diethylaminoethanol, ethanolamine, ethylenediamine, lysine, -2-hydroxyethylmorpholine, piperazine, -2-hydroxyethylpyrrolidine, triethanolamine, tromethamine. Other such salts may include salts of N-protected peptides.

“Steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone and its derivatives (e.g., hydrocortisone acetate, hydrocortisone butyrate), methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

“Non-steroidal anti-inflammatory agents” refers to a large group of agents that are aspirin-like in their action, including ibuprofen (Advil®), naproxen sodium (Aleve®), and acetaminophen (Tylenol®). Additional examples of non-steroidal anti-inflammatory agents that are useful may include, without limitation, oxicams, such as piroxicam, isoxicam, tenoxicam, sudoxicarn, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical preparation.

“Antipruritic agents” as used herein refers to those substances that reduce, eliminate or prevent itching. Suitable antipruritic agents include, without limitation, pharmaceutically acceptable salts of methdilazine and trimeprazine.

“An anti-oxidant agent” as used herein refers to a substance that inhibits oxidation or reactions promoted by oxygen or peroxides. Non-limiting examples of anti-oxidants may include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox.sup.R), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

“Chemotherapeutic agent” refers to chemicals useful in the treatment or control of a disease. Non-limiting examples of chemotherapeutic agents may include daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

“Antihistamine agent” as used herein refers to any of various compounds that counteract histamine in the body and that are used for treating allergic reactions (such as hay fever) and cold symptoms. Non-limiting examples of antihistamines may include chlorpheniramine, brompheniramine, dexchlorpheniramine, tripolidine, clemastine, diphenhydramine, promethazine, piperazines, piperidines, astemizole, loratadine and terfenadine.

“Vitamin” as used herein, refers to any of various organic substances essential in minute quantities to the nutrition of most animals act especially as coenzymes and precursors of coenzymes in the regulation of metabolic processes. Non-limiting examples of vitamins may include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta-hydroxy acids (such as salicylic acid and the like).

“Hormone” as used herein refers to natural substances produced by organs of the body that travel by blood to trigger activity in other locations or their synthetic analogs. Suitable hormones may include, but are not limited to, calciferol (Vitamin D₃) and its products, androgens, estrogens and progesterones.

“Anti-dandruff agents” as used herein refer to agents that reduce, eliminate or prevent a scurf from forming on skin, especially of the scalp, that comes off in small white or grayish scales. Exemplary anti-dandruff ingredients may include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, and ciclopiroxolamine, and mixtures thereof.

“Anti-skin atrophy actives” refers to substances effective in replenishing or rejuvenating the epidermal layer by promoting or maintaining the natural process of desquamation. Examples of antiwrinkle and antiskin atrophy actives may include retinoic acid its prodrugs and its derivatives (e.g., cis and trans) and analogues; salicylic acid and derivatives thereof, sulfur-containing D and L amino acids and their derivatives and salts, particularly the N-acetyl derivatives, a preferred example of which is N-acetyl L-cysteine; thiols, e.g., ethane thiol; alpha-hydroxy acids, e.g., glycolic acid, and lactic acid; phytic acid, lipoic acid; lysophosphatidic acid, and skin peel agents (e.g., phenol and the like). Sclerosing agents or sclerosants may be also employed. A “sclerosant” refers to an agent used as a chemical irritant injected into a vein in sclerotherapy. The most common ones are morrhuate sodium, sodium tetradecyl sulfate, laureth 9 and ethanolamine oleate.

Cleansing agents which may be useful include surfactant based cleansing agents, examples of which have been listed hereinabove. Other non-surfactant-based cleansing agents known to those of skill in the art may also be employed.

“Caustic agents” refer to substances capable of destroying or eating away epithelial tissue by chemical action. Caustic agents may be used to remove dead skin cells. For example, beta-hydroxy acids, naturally derived acids with a strong kerolytic effect, are useful for problem skin, acne or peeling.

“Hypopigmenting agents” refer to substances capable of depigmenting the skin. Suitable hypopigmenting agents include hydroquinones, mequinol, and various protease inhibitors including serine protease inhibitors, active soy and retinoic acid.

According to another aspect of the present invention, there is provided a method of preparing the topical preparation described herein. The process generally includes admixing the lipidated peptide or mixture of peptides, as described herein, and a suitable topical carrier. In cases where additional active ingredients, as detailed above, are present in the topical preparation, the process includes admixing these ingredients together with the active ingredients and the carrier. The mixing technique utilized in the process of the present invention may involve any one of the known techniques for formulating topical compositions. A variety of exemplary formulation techniques that are usable in the process of the present invention is described, for example, in Harry's Cosmeticology, Seventh Edition, Edited by J B Wilkinson and R J Moore, Longmann Scientific & Technical, 1982.

In another embodiment, the inventive composition entails adding the lipidated peptides to peptides an existing e.g., commercial, topical composition. The existing topical composition may be a pharmaceutical composition, in which case the lipidated peptide or peptides may be considered to be a “new excipient” by the U.S. F.D.A. Characteristics of new excipients can be found in the Guidance for Industry Nonclinical Studies for the Safety Evaluation of Pharmaceutical Excipients issued by the US Food and Drug Administration Center for Drug Evaluation and Research, in May, 2005, herein incorporated by reference.

According to another aspect of the present invention, there is provided a method of treating a medical, cosmetic and/or cosmeceutical condition associated with epithelial tissues. The method is effected by topically applying, an effective amount of the composition of the present invention as described above onto a surface.

As used herein the term “effective amount” refers to the amount of any of the topical preparations of the invention that result in a therapeutic or beneficial effect following its administration to a subject, an animal or human in need thereof. The effect may be curing, minimizing, preventing or ameliorating a disease or disorder, improving the physical appearance and aesthetics (e.g., skin hydration), or may have any other beneficial effect. The concentration of the substance is selected so as to exert its pharmaceutical, cosmeceutical or cosmetic effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound, composition or other active ingredient employed, the particular carrier utilized, and like factors. Daily amounts of the lipidated peptide vary generally in the range of about 0.1 mg to about 5 g.

The topical preparations may be topically applied as needed or desired. In another embodiment, the topical preparations may be topically applied between one and four times a day, (e.g., once in the morning and once in the evening). The topical application of the compositions of the present invention is preferably carried out for a time period that ranges between 1 and 30 days, more preferably for a time period of about fourteen days. Some conditions may require topical application for an indeterminate length of time.

The topical preparations may be administered to a keratinous substrate such as the epithelial surface of a subject, animal or human in need thereof. Non-limiting examples of epithelial surfaces onto which the topical preparations of the present invention may be applied include the lateral aspect of forearms, the lateral aspect of legs, elbows, feet, backhands, back, scalp, face, buttocks, the ear canal and any other skin surfaces, and any mucosal membrane described herein. Topical application also includes applying the inventive compositions orally to the gingiva.

In another embodiment, the surface is a wound surface. In chronic wounds, topical application may include applying topical preparations to a non-epithelial surface such as the dermis. In yet another embodiment, the wound surface is an open wound surface. As used herein an “open wound” is a physical trauma where the skin is lacerated, cut or punctured. As used herein, “a cut” is an injury that results in a break or opening in the skin, “a laceration” is a jagged, irregular cut, and “a puncture” is a wound made by a pointed object (like a nail, knife, or sharp tooth).

Alternatively, the compositions may be administered to the epithelial condition as a component of, for example, a bandage, adhesive, or transdermal patch. In these instances, the compositions may be an integral component of the bandage, adhesive, or transdermal patch and are thereby applied to the epithelial surface.

Application may be indirect. For example, the topical preparation may be applied to the inside of a latex glove. When the skin touches the inside of the latex glove, the composition of the invention is applied to the skin. In this embodiment, the compositions of the invention act to prevent inflammation of the skin caused, at least in part, by being enclosed in the glove.

As used herein the term “treating” includes abrogating, substantially inhibiting, slowing or reversing the progression of a condition, substantially ameliorating clinical or aesthetical symptoms of a condition, substantially preventing the appearance of clinical or aesthetical symptoms of a condition, protecting from harmful or annoying stimuli or generally promoting healthy epithelial tissue.

The term “condition” includes a variety of conditions related to skin or mucosal membranes. This term is meant to include disorders or diseases, the promotion of healthy epithelium, dry skin, and inflammation caused by any underlying mechanism or disorder.

As used herein “promotion of healthy skin” or “promoting healthy skin”, refers to providing cooling or soothing sensations, or reducing puffiness, or promoting the appearance of reduced wrinkling or puffiness. This phrase also refers to the subject's perception of his/her skin as appearing healthy or having the perception of wellness or youth.

In another embodiment, topical preparations may be applied to an epithelial tissue surface to protect the surface from exposure to environmental factors. Such factors include, but are not limited to, UV radiation, wind, hot climate extremes or cold climate extremes.

In yet another embodiment, the topical preparation may be applied to prevent wrinkles. In another embodiment, topical preparations may be applied to prevent photo-aging. In yet another embodiment, topical preparations may be administered to prevent redness or puffiness such as occurs in diaper rash.

In another embodiment, topical preparations may be used to prevent dry skin. The topical preparations may be administered to moisturize and protect the skin from the condition of dryness.

In another embodiment, topical preparations also are administered to treat a skin disorder that is already present, such as dry cracked skin. In another embodiment, topical preparations may be administered to treat irritated skin, such as occurs with diaper rash.

In another embodiment, topical preparations may be applied to treat inflammation. When inflammation is localized to the skin and mucosa, erythema (redness) occurs and can be treated by topical preparation.

Inflammation may result from a wide variety of non-limiting conditions. These conditions include, but are not limited to, a) dermatitis, including, but not limited to, atopic dermatitis, medicamentosa, contact dermatitis, seborrheic, nummular dermatitis, chronic dermatitis of hands and feet, generalized exfoliative stasis, and localized scratches; b) acne, including, but not limited to, acne vulgaris, nodulocystic acne, acne fulminans, steroid acne, acne keloidalis nuchae, chloracne, pyoderma faciale, and cysts; c) folliculitis, including, but limited to, scalp folliculitis, spa pool folliculitis, oil folliculitis, pityrosporum folliculitis, and gram negative folliculitis; d) pseudofolliculitis barbae; e) chilblains; f) miliaria (prickly heat); g) rosacea including, but not limited to, tinea rosacea, steroid rosacea and perioral dermatitis; h) eczema and psoriasis; i) bacterial infections including, but not limited to, staphylococcal diseases, staphylococcal scalded skin syndrome, erysipelas, folliculitis, furuncles, carbuncles, paronychial infections, and erythrasma; j) surgical interventions; k) crodermatitis enteropathica; l) Sweet's disease; m) amyloidosis including, but not limited to, lichen amyloidosis and macular amyloidosis; n) hives, including, but not limited to, acute generalized and chronic generalized hives and physical hives; o) erythema annulare centrifugum and annular erythema, including, but not limited to, erythema perstans, erythema gyratum perstans, erythema gyratum repens and erythema figuratum pertans; p) bachet syndrome including, but not limited to, uveitis, erythema nodosum, biotin response, dermatoses, pyoderma gangrenosum, erythema multiforme, aphthous ulcers, granulomatous cheilitis, dermitis herpetiformis, dermatomyositis, including juvenile DM and amyopathic DM, eosinophilic fascitis; q) insect bites and animal bites and stings, including, but not limited to, sea bather's eruption, seaweed dermatitis, swimmers itch, scombroid fish poisoning, scabies, popular urticaria, and cutaneous larva migrans; r) fungal infections including, but not limited to, dermatophyte infections, tinea corporis, tinea pedis, tinea unguium, tinea capitis, tinea cruris, tinea versicolor, tinea barbae, athlete's foot, and jock itch; s) yeast infections including, but not limited to, candidiasis, such as candida albicans, oral candida (thrush), candidal paronychia, and; t) parasites including, but not limited to, scabies, pediculosis including pediculosis capitis, pediculosis corporis, and pediculosis pubis; and v) viral infections including, but not limited to, herpes, including simplex lesions and zoster, chicken pox (varicella) lesions, rubeola (measles) and rubella (German measles); w) vasodilation, including, but not limited to, reye's syndrome and wound healing; x) trauma from breaks in skin; y) autoimmune conditions, including, but not limited to, cutaneous lupus erythematosus; z) bullous disease, including, but not limited to, phemphigus; aa) adverse drug reactions; bb) a immune hyper-reactivity conditions including, but not limited to, polymorphic light eruption, photosensitivity, dermographism, and erythema multiforme; cc) cancer; dd) burns; ee) wounds; ff) cysts, gg) hidradinitis suppurativa hh) cellulitis.

While topical preparations discussed herein do not necessarily treat the underlying disease or condition of the skin, topical preparations may be useful for diminishing or alleviating the disease or condition of the skin.

Additionally, topical preparations may be used in anorectal creams and suppositories to treat conditions such as a pruritus, proctitis, anal fissures, and hemorrhoids.

The topical preparations may further be used in ophthalmological preparations to treat inflammation such as that which results from corneal ulcers, radialkeratotomy, corneal transplants, epikeratophakia and other surgically induced wounds in the eye.

The topical preparation may also be used orally in the form of a mouth wash or spray to protect and accelerate the healing of injured oral tissue such as mouth sores, burns or gingivitis.

The present invention described herein has both human and veterinary utility. The term “subject” as used herein includes animals of, avian, reptilian or mammalian origin. Preferably, subjects are mammals. Even more preferably, subjects are human.

### EXAMPLES

As depicted in the Examples below, in certain exemplary embodiments, peptides and formulations of the same are prepared according to the following general procedures. It will be appreciated that, although the general methods depict the synthesis of certain peptides and formulations of the present invention, the following general methods, and other methods known to one of ordinary skill in the art, can be applied to each of the peptides and formulations disclosed herein.

### Example 1

### Method of Making Lipidated Peptides

Protein extract (100 mL, 5 mg/ml) is mixed with trypsin (100 mL, 0.1 mg/ml) or other endopeptidases (0.1 mg/ml) and incubated at 37° C. for 2 hours. The extract is further digested by exopeptidases (100 mL, 0.1 mg/ml). After digestion, the free amino group of hydrolyzed protein (˜4.6 mmole) in buffer solution is stirred with acetic anhydride (10 mL) at room temperature overnight. The protected peptide is converted to an activated ester with 2-(1H-7-Azabenzotriazol-1-yl)-1,1,3,3-tetramethyl uranium hexafluorophosphate (HATU) (1.79 g, 4.6 mmole). The active peptides are coupled to lipidated cysteine residue such as farnesyl-5-cysteine, phytyl-5-cysteine, or geranylgeranyl-5-cysteine (4.6 mmole) with N,N-diisopropylethylamine (1.2 g, 9.2 mmole) at room temperature overnight to yield 1.0 g to 1.8 g of the desired mixture of uniformly lipid modified peptides.

### Example 2

### Method of Making Lipidated Peptides

Protein extract (100 mL, 5 mg/ml) is mixed with trypsin (100 mL, 0.1 mg/ml) or other endopeptidases (0.1 mg/ml) and incubated at 37° C. for 2 hours. The resulting hydrolyzed mixture of peptides is treated with DTT (770 mg, 4.6 mmole) overnight at room temperature for reducing disulfide bonds. The free thiol content of a sample of the material is measured using a method such as reaction with Ellman's reagent (DTNB, 5,5′-dithiobis-(2-nitrobenzoic acid)). A sample of hydrolyzed protein extract (1 ml) is dissolved in 0.1 M potassium phosphate buffer at pH 7.4, and is mixed with 20 mM DTNB solution (50 μL) in the same buffer. Absorbance is observed at 412 nm at 30° C. The DTNB reagent reacts with free thiol to release 2-nitro-5-thiobenzoic acid (TNB) (1 eq.), which has a molar extinction coefficient of 14,150 M⁻¹ cm⁻¹ at 30° C. (the concentration of TNB is equal to the concentration of thiol groups in the protein sample). The peptide digest (50 g) is suspended in ethanol (200 mL) and potassium carbonate (2M aqueous) is added to adjust pH to 9.0. Phytyl-bromide (1.5 mol), farnesyl bromide, or other activated lipid is added slowly at a rate of 500 μL per minute for each mol of free thiol, with vigorous stirring to ensure uniform dispersion of the lipid in the reaction mixture. Stirring is continued for three hours after addition is complete, pH is adjusted to 6.0 with ice cold hydrochloric acid (3N), the reaction mixture is evaporated to dryness, resuspended in water (200 mL) and washed with three portions of hexane (200 mL). The salts are removed by filtering the product through a Sephadex G-10 column (equilibrated in water), and the desired product is evaporated to dryness. The resultant product is 50.0 g to 52.5 g of uniformly lipid modified peptides.

### Example 3

### Method of Making Lipidated Peptides

An amino-protected cysteine residue, N—BOC—S-farnesyl-L-cysteine (BOC—FC) (7.9970 g, 18.79 mmol) is dissolved in ethanol (37.6 mL, 2 mL/mmol) and water (9.4 mL, 0.5 mL/mmol). The mixture is titrated to pH 7 with aqueous cesium carbonate (3 mL, 2M) and the solvent is evaporated to form crude BOC—FC cesium salt, which is then dissolved in dry tetrahydrofuran (THF, 20 mL each), and is evaporated three times to yield purified BOC—FC cesium salt (10.4551 g). Separately, the Merrifield resin (8.54 g, 1 eq. resin:1.2 eq. Boc-FC) is pre-swelled in dichloromethane (DCM, 100 mL) for one hour, the DCM is rinsed with N,N-dimethylformamide (DMF, 2×100 mL) to yield an equilibrated Merrifield resin.

Purified BOC—FC cesium salt is dissolved in DMF (15 mL) is added to the equilibrated Merrifield resin in a 125 mL Erlenmeyer flask. The reaction mixture is placed in a vigorously shaking incubator at 55° C. overnight to form BOC—FC-Merrified resin. The BOC—FC-Merrifield resin is recovered by suction filtration, is washed with DMF (3×100 mL), 1:1 DMF:water (3×100 mL), DCM (3×100 mL), and methanol (3×100 mL), and is dried in a vacuum desiccator containing potassium hydroxide for 36 hours at 20-25° C. ambient room temperature to yield BOC—FC-Merrifield resin (11.0608 g).

BOC—FC-Merrifield resin (5.6020 g) is placed in a 250 mL round-bottom flask, and 1:1 trifluoroacetic acid:DCM (100 mL) is added. The flask is placed on a rotary evaporator without vacuum and the reaction is mixed for 30 minutes and the solvent is removed by suction filtration to result in crude deprotected FC-resin. Crude deprotected FC-resin is washed sequentially with DCM (3×100 mL), methanol (3×100 mL), DCM (3×100 mL), methanol (2×100 mL), pentane (100 mL), and methanol (3×100 mL), and dried overnight in a vacuum desiccator with potassium hydroxide to yield deprotected FC-resin (5.3364 g).

A single protected amino acid, e.g. Boc-L-phenylalanine (Boc-Phe, 265.3 mg, 1.0 mmol) is dissolved in DMF (2.5 mL) with HATU (418.3 mg, 1.1 mmol) and triethylamine (415.9 μL, 3 mmol) in a 250 mL Erlenmeyer flask and is swirled for 5 minutes to pre-activate the amino acid. To this mixture is added deprotected FC-resin (1 g, 0.5 mmol). The flask is covered with aluminum foil and is placed in a shaking incubator overnight at 55° C. to form Boc-Phe-FC-resin.

Boc-Phe-FC-resin is isolated from the reaction mixture by suction filtration and is washed sequentially with DMF (3×100 mL), DCM (4×100 mL), and THF (2×100 mL), then resuspended in a lithium hydroxide solution (100 mL, 0.77M; 3229.4 mg LiOH, 40 mL deionized water, 40 mL THF, 20 mL methanol). The reaction mixture is placed in a shaking incubator at 55° C. for 3 hours to cleave the desired Boc-Phe-FC from the resin. The resin, which no longer holds the product, is removed by suction filtration, and washed with water (3×100 mL) and ethyl acetate (3×100 mL). The combined washes are acidified to pH 5 with saturated aqueous ammonium chloride (25 mL), and extracted in ethyl acetate (3×100 mL). The combined organic phase is dried over sodium sulfate and is evaporated to dryness to yield the desired Boc-Phenylalanyl-FC (254.3 mg).

### Example 4

### Formulation

Uniformly lipid modified peptide is prepared substantially as described in Example 1. A topical serum is made by mixing a peptide of the present invention (50 g, 5% (w/v)), sodium hyaluronate (6250 mg, 0.625% (w/v)), chondroitin-6-sulfate sodium salt (6250 mg, 0.625% (w/v)), pantothenol (312.5 mg, 0.03125% (w/v)), Lubrajel MS (1875 mg, 0.1875% (w/v)) and glycerol (1875 mg, 0.1875% (w/v)). The mixture is dissolved in deionized water (1 L) and is mixed for 16-24 hours at 20-25° C., or until uniformly dissolved.

### EQUIVALENTS

Those skilled in the art will recognize, or be able to ascertain using no more than routine experimentation, that while the invention herein has been described with reference to particular embodiments, it is to be understood that these embodiments are merely illustrative of the principles and applications of the present invention and other embodiments may achieve the same results. It is therefore to be understood that numerous modifications may be made to the illustrative embodiments and that other arrangements may be devised without departing from the spirit and scope of the present invention as defined by the appended claims. The preceding examples may be repeated with similar success by substituting the generically or specifically described reactants and/or operating conditions used.

In the claims articles such as “a,” “an,” and “the” may mean one or more than one unless indicated to the contrary or otherwise evident from the context. Claims or descriptions that include “or” between one or more members of a group are considered satisfied if one, more than one, or all group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any linking agent, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

1. A topical composition comprising a polypeptide containing a lipidated cysteine residue and an acceptable carrier, wherein the polypeptide is present in an amount of 0.01 to about 10% by weight of the composition.

2-30. (canceled)

31. A method of treating an epithelial-related condition, said method comprising topically applying onto a surface of a mammal, including a human, in need thereof a pharmaceutically effective amount of a composition comprising at least one polypeptide and a carrier.

32. The method of claim 31, wherein the polypeptide has a C-terminal amino acid sequence selected from the group consisting of SEQ ID NOs 1-113.

33. The method of claim 31, wherein the polypeptide has a C-terminal amino acid sequence identical to a sequence selected from SEQ ID NOs 1-113 except that at least one amino acid C-terminal to the lipidated cysteine is removed.

34. The method of claim 31, wherein the polypeptide has a C-terminal amino acid sequence identical to a sequence selected from the group consisting of SEQ ID NOs 5, 6, 31, 32, 38, 42, 50, 53, 59, 64, 65, 67, 71, 80, 81, 83, 84, 89, 95, 105 and combinations thereof.

35. The method according to claim 31, wherein the carrier is at least one agent selected from the group consisting essentially of a moisturizing agent, a pH adjusting agent, a deodorant agent, a thickener, a solubilizing agent, a penetration enhancer, an anti-irritant, a colorant and a surfactant.

36. The method according to claim 31, wherein the carrier of the composition is an oral carrier selected from the group consisting of a mouthwash, a rinse, and an oral spray.

37. The method according to claim 36, wherein the oral carrier has a pH from about 5 to about 6.5.

38. The method according to claim 36, wherein the oral carrier further comprises at least one agent selected from the group consisting of a fluorine-providing compound, a sweetening agent, a coloring agent, a moisturizer, and an emulsifier.

39. The method according to claim 31, wherein the carrier is a delayed release carrier.

40. The method according to claim 39, wherein the delayed release carrier is a carrier selected from the group consisting of a liposome, a microsponge, a microsphere and a microcapsule.

41. The method according to claim 31, further comprising an active ingredient.

42. The method according to claim 41 wherein the active ingredient is at least one ingredient selected from the group consisting of a protective agent, an emollient, an astringent, an irritant, a keratolytic agent, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a vitamin, a hormone, an anti-dandruff agent, an anti-wrinkle agent, an anti-skin atrophy agent, a sclerosing agent, a cleansing agent, a caustic agent and a hypo-pigmenting agent.

43. The method according to claim 42, wherein the protective agent is selected from the group consisting of an adsorbent, a demulcent and a desiccant.

44. The method according to claim 42, wherein the irritant is a rubefacient.

45-66. (canceled)

67. A method of promoting healthy skin in a subject, including a human, in need thereof, said method comprising topically applying onto a surface of a subject, including a human, a cosmetically effective amount of a composition comprising: at least one polypeptide; and a carrier.

68. The method of claim 67, wherein the polypeptide has a C-terminal amino acid sequence selected from the group consisting of SEQ ID NOs 1-113.

69. The method of claim 67, wherein the polypeptide has a C-terminal amino acid sequence identical to a sequence selected from SEQ ID NOs 1-113 except that at least one amino acid C-terminal to the lipidated cysteine is removed.

70. The method according to claim 67, wherein the surface is an epithelial surface.

71. The method according to claim 67, wherein the surface is a mucosal membrane.

72. The method according to claim 67, wherein the surface is selected from the group consisting of a bandage and a transdermal patch.

73. A method of preparing a topical composition, the method comprising admixing at least one polypeptide and a carrier to form a powder, an oil, a cream, a gel, a bandage, and wherein the topical composition is formulated to deliver an amount of the at least one polypeptide effective to inhibit inflammation when applied topically.

74. The method of claim 73, wherein the polypeptide has a C-terminal amino acid sequence selected from the group consisting of SEQ ID NOs 1-113.

75. The method of claim 73, wherein the polypeptide has a C-terminal amino acid sequence identical to a sequence selected from SEQ ID NOs 1-113 except that at least one amino acid C-terminal to the lipidated cysteine is removed.

76. The method of claim 73, wherein the polypeptide has a C-terminal amino acid sequence identical to a sequence selected from the group consisting of SEQ ID NOs 5, 6, 31, 32, 38, 42, 50, 53, 59, 64, 65, 67, 71, 80, 81, 83, 84, 89, 95, 105 and combinations thereof.


SEQUENCE LISTING (113 sequences)

SEQ ID NO: 1 - protein, 24 aa, Artificial sequence
  Genbank Accession Number P51160
GGAEKAAEDSGGGDDKKSKTCLML

SEQ ID NO: 2 - protein, 24 aa, Artificial sequence
  Genbank Accession Number P35913
AAKKVGTEICNGGPAPKSSTCCIL

SEQ ID NO: 3 - protein, 24 aa, Artificial sequence
  Genbank Accession Number P10114
IVRQMNYAAQPDKDDPCCSACNIQ

SEQ ID NO: 4 - protein, 24 aa, Artificial sequence
  Genbank Accession Number P17964
IVRQMNYAAQPNGDEGCCSACVIL

SEQ ID NO: 5 - protein, 24 aa, Artificial sequence
  Genbank Accession Number P10113
LVRQINRKTPVEKKKPKKKSCLLL

SEQ ID NO: 6 - protein, 24 aa, Artificial sequence
  Genbank Accession Number P09526
LVRQINRKTPVPGKARKKSSCQLL

SEQ ID NO: 7 - protein, 24 aa, Artificial sequence
  Genbank Accession Number P31689
RHYNGEAYEDDEHHPRGGVQCQTS

SEQ ID NO: 8 - protein, 24 aa, Artificial sequence
  Genbank Accession Number Q8WW22
WRQHREAYEEDEDGPQAGVQCQTA

SEQ ID NO: 9 - protein, 24 aa, Artificial sequence
  Genbank Accession Number Q8N5D0
LSSGGAGASDDEDSSEGQVQCRPS

SEQ ID NO: 10 - protein, 24 aa, Artificial sequence
  Genbank Accession Number Q9Y272
DLMYIREKASAGSQAKDKERCVIS

SEQ ID NO: 11 - protein, 24 aa, Artificial sequence
  Genbank Accession Number Q96D21
DLKYIKAKVLREGQARERDKCTIQ

SEQ ID NO: 12 - protein, 24 aa, Artificial sequence
  Genbank Accession Number P17081
ILTPKKHTVKKRIGSRCINCCLIT

SEQ ID NO: 13 - protein, 24 aa, Artificial sequence
  Genbank Accession Number P01121
TATRAALQKRYGSQNGCINCCKVL

SEQ ID NO: 14 - protein, 24 aa, Artificial sequence
  Genbank Accession Number AAB40597.1
EKYRPKMRLRFKDSNGHRNNCCIQ

SEQ ID NO: 15 - protein, 24 aa, Artificial sequence
  Genbank Accession Number AAB40598.1
LYLEKYRPKMRLRFRDTNGHCCVQ

SEQ ID NO: 16 - protein, 24 aa, Artificial sequence
  Genbank Accession Number NP_116000.1
EKYRPKQRLRFKDPHTHKTRCCVM

SEQ ID NO: 17 - protein, 24 aa, Artificial sequence
  Genbank Accession Number Q15835
MPDDMKGISGGSSSSSKSGMCLVS

SEQ ID NO: 18 - protein, 24 aa, Artificial sequence
  Genbank Accession Number Q8WTQ7
LNDPNRPTGCEEGNSSKSGVCLLL

SEQ ID NO: 19 - protein, 24 aa, Artificial sequence
  Genbank Accession Number P17082
QECPPSPEPTRKEKDKKGCHCVIF

SEQ ID NO: 20 - protein, 24 aa, Artificial sequence
  Genbank Accession Number P10301
EQELPPSPPSAPRKKGGGCPCVLL

SEQ ID NO: 21 - protein, 24 aa, Artificial sequence
  Genbank Accession Number Q9UKC9
FAPVTPPTAVAGSGQRLCRCCVIL

SEQ ID NO: 22 - protein, 24 aa, Artificial sequence
  Genbank Accession Number Q96IG2
FAPVTPPPSVGGSRQRFCRCCIIL

SEQ ID NO: 23 - protein, 24 aa, Artificial sequence
  Genbank Accession Number P01112
HKLRKLNPPDESGPGCMSCKCVLS

SEQ ID NO: 24 - protein, 24 aa, Artificial sequence
  Genbank Accession Number P01111
YRMKKLNSSDDGTQGCMGLPCVVM

SEQ ID NO: 25 - protein, 24 aa, Artificial sequence
  Genbank Accession Number Q13072
MKEESPVVSWRLEPEDGTALCFIF

SEQ ID NO: 26 - protein, 24 aa, Artificial sequence
  Genbank Accession Number Q86Y27
MKEESPVVSWRLEPEDGTALCFIF

SEQ ID NO: 27 - protein, 24 aa, Artificial sequence
  Genbank Accession Number Q96PZ7
TNLKPTEAKAVRFDTTLNTVCTVV

SEQ ID NO: 28 - protein, 24 aa, Artificial sequence
  Genbank Accession Number Q7Z407
TNAKSVEGKAVRFDPNLNTVCTMV

SEQ ID NO: 29 - protein, 24 aa, Artificial sequence
  Genbank Accession Number P32455
SRIMKNEIQDLQTKMRRRKACTIS

SEQ ID NO: 30 - protein, 24 aa, Artificial sequence
  Genbank Accession Number O75781
PEATTSDPQDLDMKKHRCKCCSIM

SEQ ID NO: 31 - protein, 24 aa, Artificial sequence
  Genbank Accession Number Q92730
HLPSRSELISSTFKKEKAKSCSIM

SEQ ID NO: 32 - protein, 24 aa, Artificial sequence
  Genbank Accession Number P52199
PSRPELSAVATDLRKDKAKSCTVM

SEQ ID NO: 33 - protein, 24 aa, Artificial sequence
  Genbank Accession Number NP_660156.1
LNIDGKRSGKQKRTDRVKGKCTLM

SEQ ID NO: 34 - protein, 24 aa, Artificial sequence
  Genbank Accession Number NP_060064.2
LQIDGKKSKQQKRKEKLKGKCVIM

SEQ ID NO: 35 - protein, 24 aa, Artificial sequence
  Genbank Accession Number O95661
LQEPEKKSQMPNTTEKLLDKCIIM

SEQ ID NO: 36 - protein, 24 aa, Artificial sequence
  Genbank Accession Number P01116
YRLKKISKEEKTPGCVKIKKCIIM

SEQ ID NO: 37 - protein, 24 aa, Artificial sequence
  Genbank Accession Number P01118
KHKEKMSKDGKKKKKKSKTKCVIM

SEQ ID NO: 38 - protein, 24 aa, Artificial sequence
  Genbank Accession Number P50152
EDPLVKGIPEDKNPFKEKGSCVIS

SEQ ID NO: 39 - protein, 24 aa, Artificial sequence
  Genbank Accession Number Q08447
DPLVKGIPEDKNPFKELKGGCVIS

SEQ ID NO: 40 - protein, 24 aa, Artificial sequence
  Genbank Accession Number O14610
NDPFLKGIPEDKNPFKEKGGCLIS

SEQ ID NO: 41 - protein, 24 aa, Artificial sequence
  Genbank Accession Number Q9P2W3
KDPFLNPDLMKNNPWVEKGKCTIL

SEQ ID NO: 42 - protein, 24 aa, Artificial sequence
  Genbank Accession Number Q9UBI6
SDPLLIGIPTSENPFKDKKTCIIL

SEQ ID NO: 43 - protein, 24 aa, Artificial sequence
  Genbank Accession Number O60262
NDPLLVGVPASENPFKDKKPCIIL

SEQ ID NO: 44 - protein, 24 aa, Artificial sequence
  Genbank Accession Number Q9Y3K8
HDPLLTGVSSSTNPFRPQKVCSFL

SEQ ID NO: 45 - protein, 24 aa, Artificial sequence
  Genbank Accession Number P30670
HDPLLTGVSSSTNPFRPQKVCSFL

SEQ ID NO: 46 - protein, 24 aa, Artificial sequence
  Genbank Accession Number P59768
DPLLTPVPASENPFREKKFFCAIL

SEQ ID NO: 47 - protein, 24 aa, Artificial sequence
  Genbank Accession Number P29798
DPLITPVPTSENPFREKKFFCALL

SEQ ID NO: 48 - protein, 24 aa, Artificial sequence
  Genbank Accession Number P50150
DPLIIPVPASENPFREKKFFCTIL

SEQ ID NO: 49 - protein, 24 aa, Artificial sequence
  Genbank Accession Number Q9UK08
DPLVTPVPAAENPFRDKRLFCVLL

SEQ ID NO: 50 - protein, 24 aa, Artificial sequence
  Genbank Accession Number P50151
KDALLVGVPAGSNPFREPRSCALL

SEQ ID NO: 51 - protein, 24 aa, Artificial sequence
  Genbank Accession Number Q96I34
PPLLKLTAPAVEAPVERRPCCLLM

SEQ ID NO: 52 - protein, 24 aa, Artificial sequence
  Genbank Accession Number Q96T49
PPLLKFKAPIEEMEEKVHGCCRIS

SEQ ID NO: 53 - protein, 24 aa, Artificial sequence
  Genbank Accession Number P20700
VVVEEELFHQQGTPRASNRSCAIM

SEQ ID NO: 54 - protein, 24 aa, Artificial sequence
  Genbank Accession Number Q03252
EFGEEDLFHQQGDPRTTSRGCYVM

SEQ ID NO: 55 - protein, 24 aa, Artificial sequence
  Genbank Accession Number P15154
FDEAIRAVLCPPPVKKRKRKCLLL

SEQ ID NO: 56 - protein, 24 aa, Artificial sequence
  Genbank Accession Number O95916
FDEAIRAVLCPPPVKKRKRKCLQL

SEQ ID NO: 57 - protein, 24 aa, Artificial sequence
  Genbank Accession Number P60763
FDEAIRAVLCPPPVKKPGKKCTVF

SEQ ID NO: 58 - protein, 24 aa, Artificial sequence
  Genbank Accession Number P15153
FDEAIRAVLCPQPTRQQKRACSLL

SEQ ID NO: 59 - protein, 24 aa, Artificial sequence
  Genbank Accession Number P35238
VFAEAVRAVLNPTPIKRGRSCILL

SEQ ID NO: 60 - protein, 24 aa, Artificial sequence
  Genbank Accession Number P60953
VFDEAILAALEPPETQPKRKCCIF

SEQ ID NO: 61 - protein, 24 aa, Artificial sequence
  Genbank Accession Number P06749
VFEMATRAALQARRGKKKSGCLVL

SEQ ID NO: 62 - protein, 24 aa, Artificial sequence
  Genbank Accession Number P08134
VFEMATRAGLQVRKNKRRRGCPIL

SEQ ID NO: 63 - protein, 24 aa, Artificial sequence
  Genbank Accession Number P46019
MTYLTRAVASYLQELLPNSGCQMQ

SEQ ID NO: 64 - protein, 24 aa, Artificial sequence
  Genbank Accession Number P46020
MTYLSKAAATYVQEFLPHSICAMQ

SEQ ID NO: 65 - protein, 24 aa, Artificial sequence
  Genbank Accession Number P52198
LSGRPDRGNEGEIHKDRAKSCNLM

SEQ ID NO: 66 - protein, 24 aa, Artificial sequence
  Genbank Accession Number P51398
EGKKELLFLSNANPSLLERHCAYL

SEQ ID NO: 67 - protein, 24 aa, Artificial sequence
  Genbank Accession Number P16499
AAGNQPGGNPSPGGATTSKSCCIQ

SEQ ID NO: 68 - protein, 24 aa, Artificial sequence
  Genbank Accession Number P40855
LNFDLDALNLSGPPGASGEQCLIM

SEQ ID NO: 69 - protein, 24 aa, Artificial sequence
  Genbank Accession Number P02545
LVTRSYLLGNSSPRTQSPQNCSIM

SEQ ID NO: 70 - protein, 24 aa, Artificial sequence
  Genbank Accession Number P01730
IKRLLSEKKTCQCPHRFQKTCSPI

SEQ ID NO: 71 - protein, 24 aa, Artificial sequence
  Genbank Accession Number AAF81404.1
RISKEIQRQQALQSQNSSTICSVS

SEQ ID NO: 72 - protein, 24 aa, Artificial sequence
  Genbank Accession Number Q9HBH0
AAKVALSALKKAQRQKKRRLCLLL

SEQ ID NO: 73 - protein, 24 aa, Artificial sequence
  Genbank Accession Number P32019
HQKLDMTEKKKAQEFIHQFLCNPL

SEQ ID NO: 74 - protein, 24 aa, Artificial sequence
  Genbank Accession Number O95858
GLLGPGAKPSVEAAGTGCCLCYPN

SEQ ID NO: 75 - protein, 24 aa, Artificial sequence
  Genbank Accession Number P43119
TQQSSGSAVGTSSKAEASVACSLC

SEQ ID NO: 76 - protein, 24 aa, Artificial sequence
  Genbank Accession Number Q92737
VRARPAHPALRLQGALHPARCSLM

SEQ ID NO: 77 - protein, 24 aa, Artificial sequence
  Genbank Accession Number CAE51163.1
NQQFPWVDPGPRPPRLPLLECTPQ

SEQ ID NO: 78 - protein, 24 aa, Artificial sequence
  Genbank Accession Number Q9H4E5
ILTIFHPKKKKKRCSEGHSCCSII

SEQ ID NO: 79 - protein, 24 aa, Artificial sequence
  Genbank Accession Number P49454
KRGRLVPSPKAGLESKGSENCKVQ

SEQ ID NO: 80 - protein, 24 aa, Artificial sequence
  Genbank Accession Number Q15382
RRIILEAEKMDGAASQGKSSCSVM

SEQ ID NO: 81 - protein, 24 aa, Artificial sequence
  Genbank Accession Number O15255
PPRDLSPSARPISSPPPETSCVLA

SEQ ID NO: 82 - protein, 24 aa, Artificial sequence
  Genbank Accession Number Q02224
ESVDSQPGPWHASSGKDVPECKTQ

SEQ ID NO: 83 - protein, 24 aa, Artificial sequence
  Genbank Accession Number P09543
YGKGKPVPTQGSRKGGALQSCTII

SEQ ID NO: 84 - protein, 24 aa, Artificial sequence
  Genbank Accession Number P48448
YPPYTDWNQQLLRWGMGSQSCTLL

SEQ ID NO: 85 - protein, 24 aa, Artificial sequence
  Genbank Accession Number Q9H078
DKDSKTRRLDIRAPLHPEKVCNTI

SEQ ID NO: 86 - protein, 24 aa, Artificial sequence
  Genbank Accession Number NP_115971.2
KKMADTSSMDEDFESDYKKYCVLQ

SEQ ID NO: 87 - protein, 24 aa, Artificial sequence
  Genbank Accession Number P45381
EKKEAFAKTTKLTLNAKSIRCCLH

SEQ ID NO: 88 - protein, 24 aa, Artificial sequence
  Genbank Accession Number O14807
KKKKKTKWRGDRATGTHKLQCVIL

SEQ ID NO: 89 - protein, 24 aa, Artificial sequence
  Genbank Accession Number P32456
KRLQKDIWDIQMRSKSLEPICNIL

SEQ ID NO: 90 - protein, 24 aa, Artificial sequence
  Genbank Accession Number P11233
SKEKNGKKKRKSLAKRIRERCCIL

SEQ ID NO: 91 - protein, 24 aa, Artificial sequence
  Genbank Accession Number AAH40076.1
SKSRTPDKMKNLSKSWWKKYCCFV

SEQ ID NO: 92 - protein, 24 aa, Artificial sequence
  Genbank Accession Number O94955
NMYLKQLAEYRKYIHSRKCRCLVM

SEQ ID NO: 93 - protein, 24 aa, Artificial sequence
  Genbank Accession Number P34949
ANESVSLKLTEPKDLLIFRACCLL

SEQ ID NO: 94 - protein, 24 aa, Artificial sequence
  Genbank Accession Number Q9Y6Q2
VEIEKKWIKIDGEDPDKIGDCITQ

SEQ ID NO: 95 - protein, 24 aa, Artificial sequence
  Genbank Accession Number P43353
PQSPRRLRMLLVAMEAQGCSCTLL

SEQ ID NO: 96 - protein, 24 aa, Artificial sequence
  Genbank Accession Number Q15669
RTAVNQARRRNRRRLFSINECKIF

SEQ ID NO: 97 - protein, 24 aa, Artificial sequence
  Genbank Accession Number O95236
AQELEENLMELTQIYQRLNPCHTH

SEQ ID NO: 98 - protein, 24 aa, Artificial sequence
  Genbank Accession Number Q13286
SDTLGISLSGLLALPLHDFLCQLS

SEQ ID NO: 99 - protein, 24 aa, Artificial sequence
  Genbank Accession Number Q93100
TKAVMNLLLEGEVKPNNDDPCLIS

SEQ ID NO: 100 - protein, 24 aa, Artificial sequence
  Genbank Accession Number Q96PP8
NRSLLSELQHAQRTVNNDDPCVLL

SEQ ID NO: 101 - protein, 24 aa, Artificial sequence
  Genbank Accession Number Q9Y2C3
IKPRTLLDYWQALENSRGEDCPPV

SEQ ID NO: 102 - protein, 24 aa, Artificial sequence
  Genbank Accession Number Q14642
FLAFRIMPGAGKPHAHVHKCCVVQ

SEQ ID NO: 103 - protein, 24 aa, Artificial sequence
  Genbank Accession Number O43900
SLSLPRDSRAGMPRQARDKNCIVA

SEQ ID NO: 104 - protein, 24 aa, Artificial sequence
  Genbank Accession Number P00387
PMIQYACLPNLDHVGHPTERCFVF

SEQ ID NO: 105 - protein, 24 aa, Artificial sequence
  Genbank Accession Number Q96M98
RYGGENAFINIKYVVPTYESCLLN

SEQ ID NO: 106 - protein, 24 aa, Artificial sequence
  Genbank Accession Number O60635
VAAGIGGLELAAMIVSMYLYCNLQ

SEQ ID NO: 107 - protein, 24 aa, Artificial sequence
  Genbank Accession Number P59022
PFCAEPCSRVTVCHLQAVPVCMPV

SEQ ID NO: 108 - protein, 24 aa, Artificial sequence
  Genbank Accession Number Q9BUV8
LFMVCVADSFTTGHLDHLLHCHPL

SEQ ID NO: 109 - protein, 24 aa, Artificial sequence
  Genbank Accession Number Q9UBV7
GAPCTVLNIMLDCDKTATPWCTFS

SEQ ID NO: 110 - protein, 24 aa, Artificial sequence
  Genbank Accession Number Q16589
SPPSDQECTFFFNFKVAQTLCFPS

SEQ ID NO: 111 - protein, 24 aa, Artificial sequence
  Genbank Accession Number Q8WVZ1
AAASWMRLASASCRAKPWAVCFPS

SEQ ID NO: 112 - protein, 24 aa, Artificial sequence
  Genbank Accession Number O00212
AEVALSSRGRNFWRRITQGFCVVT

SEQ ID NO: 113 - protein, 24 aa, Artificial sequence
  Genbank Accession Number Q9NQX7
AKNCNAIRHFENTFVVETLICGVV